(19) United States
(12) Patent Application Publication
(10) Pub. No.: US 2023/0063611 A1
(43) Pub. Date: Mar. 2, 2023

(54) POROUS BIOCOMPATIBLE IMPLANT WITH EXCELLENT OSSEOINTEGRATION AND METHOD FOR MANUFACTURING SAME

(71) Applicants: Inn Kyu KANG, Daegu (KR); JEIL MEDICAL CORPORATION, Seoul (KR)

(72) Inventors: Inn Kyu KANG, Daegu (KR); Hun KIM, Daegu (KR); Yang Ho LEE, Daegu (KR)

(21) Appl. No.: 17/693,904

(22) Filed: Mar. 14, 2022

(30) Foreign Application Priority Data
Aug. 26, 2021 (KR) 10-2021-0113154

(51) Int. Cl. A61L 27/56; A61L 27/18; A61L 27/34

(52) U.S. Cl. CPC A61L 27/56; A61L 27/18; A61L 27/34; A61L 2420/02; A61L 2430/38

(57) ABSTRACT

The present invention relates to a porous biocompatible implant and a method for manufacturing the same, and more specifically to a porous biocompatible implant in which osseointegration is excellent, no dissociation from the implant occurs, the inflammatory response caused by metals or bacteria can be minimized, and it is possible to accelerate bone formation, while having excellent mineralized bone formation performance, and a method for manufacturing the same. In addition, the porous biocompatible implant of the present invention can be widely used in clinical practices such as dentistry and orthopedic surgery.

### CROSS REFERENCE TO RELATED APPLICATION

This application claims priority to and the benefit of Korean Patent Application No. 10-2021-0113154, filed on Aug. 26, 2021, the disclosure of which is incorporated herein by reference in its entirety.

### FIELD OF THE DISCLOSURE

The present invention relates to an implant for dentistry and/or orthopedic surgery, and more specifically to a porous implant with excellent osseointegration and excellent mechanical properties and a method for manufacturing the same.

### DESCRIPTION OF RELATED ART

In general, medical biocompatible implants are used to permanently implant spinal fixation prostheses, interspecies correction prostheses, artificial joints and the like, and must use biocompatible materials that are very stable and biocompatible with human tissue. In addition, there should be no side effects or other chemical and biochemical reactivity, and the mechanical strength should be very high so as not to be deformed and destroyed even when repeated loads and instantaneous pressures are applied, and these are devices for medical purposes that must have a very high binding strength with living tissue, particularly, bone tissue.

These implants are removed from the bone tissue after being implanted in the bone tissue to function permanently or only for a period of time performing the intended function, and during implantation, it is common to perform various processing on the surface of the implant in order to increase the binding strength with the bone tissue. Examples of such processing include roughening processing to increase a specific surface area with bone tissue, or coating a component similar to bone tissue, for example, a component such as hydroxyapatite on the surface of the implant.

However, even when the surface of the implant is processed to have a high binding strength with the bone tissue, the solutions to side effects such as inflammation occurring in the surrounding tissue after the implant is placed are still insufficient until now. In particular, the act of generating orthodontic force, such as pulling an orthodontic wire as a support for orthodontic implants after being implanted such as orthodontic micro-implants, forms gaps between the implant and the alveolar bone or surrounding tissues, and inflammatory causes such as bacteria and the like penetrate into these gaps and cause inflammation, which can cause failure of the implant placement procedure.

In addition, conventional implant materials are generally implemented with biocompatible materials such as titanium to minimize in vivo side effects, and in the case of commercially available titanium implants, tissue inflammation is induced, and such tissue inflammation creates gaps between the implant and the implanted bone and/or gum, and by making the gaps more spaced apart, there is a problem that causes inflammation due to additional bacterial infiltration. Moreover, in the case of conventional implants, as the osseointegration ability is lowered, the phenomenon of detachment from the affected area after the procedure occurs, or in the case of surface-treated implants, there is a problem in that safety is reduced in terms of permanent implantation due to dissociation with the implant and the like.

Accordingly, the situation is that there is an urgent need for research on implants which are capable of simultaneously exhibiting all of the effects of having excellent osseointegration, not causing dissociation with implants, minimizing inflammatory reactions caused by metals or bacteria, accelerating bone formation, and having excellent mineralized bone formation performance.

### SUMMARY OF THE INVENTION

The present invention has been devised in view of the above points, and it is an object of the present invention to provide a porous biocompatible implant which is capable of simultaneously exhibiting all of the effects of having excellent osseointegration, not causing dissociation with the implant, minimizing inflammatory reactions caused by metals or bacteria, accelerating bone formation, and having excellent mineralized bone formation performance, and a method for manufacturing the same.

In order to solve the aforementioned problems, the present invention relates to a porous biocompatible implant, which includes a collagen layer which is immobilized on at least a portion of the surface of a porous polyether ether ketone (PEEK) implant.

According to an exemplary embodiment of the present invention, the porous PEEK implant may be formed with a plurality of pores in a single layer on the surface.

According to an exemplary embodiment of the present invention, the porous PEEK implant may be formed with a single pore layer having a thickness of 0.5 µm to 3 µm from the surface.

According to an exemplary embodiment of the present invention, the porous biocompatible implant may have a diameter of pores formed on the surface of less than 1,000 nm.

According to an exemplary embodiment of the present invention, the collagen layer may be implemented through a stem on fibers formed by gathering a plurality of collagen molecules.

According to an exemplary embodiment of the present invention, the collagen layer may be immobilized to the porous biocompatible implant through polydopamine.

According to an exemplary embodiment of the present invention, the collagen layer may be immobilized to the porous biocompatible implant by substitution with an amine group of the polydopamine.

According to an exemplary embodiment of the present invention, the collagen layer may be formed by using salt-precipitated compression-concentrated liquid collagen derived from pig skin.

According to an exemplary embodiment of the present invention, when the surface of the porous biocompatible implant to which collagen and polydopamine are not bound is measured with an X-ray photoelectron spectrometer, nitrogen (N) may be included at 4.00 to 6.00 atom% based on 100% of the total composition of carbon (C), oxygen (O), nitrogen (N) and sulfur (S) in the composition of the surface.

According to an exemplary embodiment of the present invention, when the surface of the porous biocompatible implant is measured with an X-ray photoelectron spectrometer, nitrogen (N) may be included at 15.50 atom% or more based on 100% of the total composition of carbon (C), oxygen (O), nitrogen (N) and sulfur (S) in the composition of the surface.

In addition, the present invention relates to a method for manufacturing the above-described porous biocompatible implant, which may be manufactured by performing a process including step 1 of manufacturing a porous PEEK implant by forming a plurality of pores on the surface of a PEEK implant; step 2 of manufacturing a polydopamine-coated porous PEEK implant by coating and drying the porous PEEK implant with a dopamine coating solution; and step 3 of immobilizing a porous collagen layer on at least a portion of the surface of the polydopamine-coated porous PEEK implant.

According to an exemplary embodiment of the present invention, the porous PEEK implant of step 1 may be manufactured by performing step 1-1 of performing an immersion process by immersing the PEEK implant in a mixed acid solution for 1 minute to 5 minutes; step 1-2 of performing stirring at a speed of 200 to 500 rpm for 3 minutes to 10 minutes, after the PEEK implant subjected to the immersion process is placed into distilled water; step 1-3 of washing the PEEK implant for which step 1-2 has been performed with a high-pressure washer by applying water at a water pressure of 120 to 170 bar for 1 minute to 30 minutes; and step 1-4 of drying the PEEK implant for which step 1-3 has been performed, after performing ultrasonic cleaning under an ultrasonic intensity of 15 to 50 KHz (method 1).

According to an exemplary embodiment of the present invention, the porous PEEK implant of step 1 may be manufactured by performing step 1-1 of performing an immersion process under ultrasonication by immersing the PEEK implant in a mixed acid solution for 1 minute to 5 minutes; step 1-2 of performing stirring at a speed of 200 to 500 rpm for 3 minutes to 10 minutes, after the PEEK implant subjected to the immersion process is placed into distilled water; and step 1-3 of drying the PEEK implant for which step 1-2 has been performed, after performing ultrasonic cleaning under an ultrasonic intensity of 50 to 80 KHz (method 2).

According to an exemplary embodiment of the present invention, the dopamine coating solution may include dopamine hydrochloride and a Tris buffer aqueous solution having a pH of 8.0 to 9.0.

According to an exemplary embodiment of the present invention, the dopamine coating solution may include dopamine hydrochloride at a concentration of 0.20 to 0.40 mg/mL.

According to an exemplary embodiment of the present invention, in step 3, the polydopamine-coated porous PEEK implant may be coated with a liquid collagen solution, washed and dried to form a collagen layer.

According to an exemplary embodiment of the present invention, the liquid collagen solution may include 0.0005 to 0.05 wt.%of collagen, 0.100 to 0.300 wt.%of EDC ((1-ethyl-3-(3-dimethyl aminopropyl)carbodiimide), 0.100 to 0.300 wt.%of NHS (N-hydroxysuccinimide) and the remaining amount of water.

In the present invention, since the porous biocompatible implant has pores having a uniform size on the surface of the implant, the adhesion to the collagen layer is excellent due to an increase in the specific surface area. In addition, since it can immobilize a large amount of collagen, it has excellent osseointegration, and no dissociation from the implant occurs, and it is possible to minimize inflammatory reactions caused by metals or bacteria and to accelerate bone formation, and at the same time, it has excellent mineralized osteogenic properties. In addition, since the pores formed on the surface are not formed three-dimensionally, but are formed only on a portion of the surface of the implant in two dimensions, the mechanical properties of the PEEK implant itself are hardly affected, and it can be widely used as a biocompatible implant for dentistry and/or orthopedic surgery.

### BRIEF DESCRIPTION OF THE DRAWINGS

FIG. 1 is a mimetic diagram of manufacturing a porous biocompatible implant with excellent osseointegration according to an exemplary embodiment of the present invention.

FIG. 2 is an SEM measurement image of the surface of the PEEK implant manufactured in Comparative Preparation Example 1.

FIGS. 3 (A and B) are SEM images at high magnification and low magnification of the porous PEEK implant manufactured in Preparation Example 1, respectively.

FIGS. 4 (A-C) are SEM images measured at different magnifications for the porous PEEK implant manufactured in Preparation Example 2, respectively.

FIGS. 5 (A-D) are SEM images measured at different magnifications for the porous PEEK implant manufactured by using sulfuric acid in Comparative Preparation Example 2, respectively.

FIG. 6 is an SEM surface tomography image of the porous PEEK implant manufactured in Comparative Preparation Example 2, and it is an image after etching with sulfuric acid.

FIG. 7 is an image showing an SEM surface tomography image of the porous PEEK implant manufactured in Preparation Example 2, and it is an image after etching with a mixed acid.

FIGS. 8 (A and B) are SEM measurement images of the porous PEEK-D and porous PEEK-DC manufactured in Example 1, respectively.

FIG. 9 (A) is an electron microscope image before pore formation, (B) is an electron microscope image after pore formation, and (C) is an electron microscope image after 3 hours of incubation of osteoblasts on the surface of a collagen-immobilized PEEK implant.

FIG. 10 (A) is a fluorescence image obtained by staining calcium produced by incubating osteoblasts on a non-porous PEEK implant for 7 days with Alizarin Red S, (B) on a porous PEEK implant, and (C) on a collagen-immobilized PEEK implant (porous PEEK-DC), respectively.

### DETAILED DESCRIPTION OF THE INVENTION

As used herein, the term “porous biocompatible implant” refers to an implant including polydopamine on the surface of a PEEK implant and a collagen layer immobilized to the surface of the implant by the polydopamine.

Hereinafter, the present invention will be described in more detail.

The porous biocompatible implant according to the present invention is implemented by including a collagen layer immobilized on at least a portion of the surface of a porous polyether ether ketone (PEEK) implant.

First, the porous PEEK implant will be described.

The implant according to the present invention is formed of polyether ether ketone, and while it can solve the problems of corrosion and metal ion release, which are problems of conventional metal-based (e.g., titanium, etc.) implants, it can simultaneously exhibit all of the excellent effects of having excellent osseointegration, accelerating bone formation and having excellent mineralized bone formation.

In addition, the porous PEEK implant is formed by etching the surface of the PEEK implant to form a plurality of pores in a two-dimensional structure, and as such, by forming pores in a single-layer structure only on the surface of the implant without forming pores in a three-dimensional structure, it is possible to increase the collagen immobilization rate and adhesion rate while maintaining excellent mechanical properties of the implant. For a preferred example, the porous PEEK implant may be formed with a pore layer having a thickness (or depth) of 0.5 µm to 3 µm from the surface, preferably, 1.0 µm to 2.5 µm, and more preferably, 1.5 µm to 2 µm. In this case, if the pore layer thickness is less than 0.5 µm, the effect of increasing the collagen adhesion rate and the immobilization rate due to pore formation may be insignificant, and if the pore layer thickness is more than 3.0 µm, there may be a problem in that the mechanical strength is reduced, and thus, preferably, the pore layer is formed from the surface to a thickness (or depth) within the above range.

In addition, the pores are uniformly formed on the surface of the porous PEEK implant to which a collagen layer is not immobilized on the surface, and the diameter of the pores formed on the surface may be formed in a range of less than 1,000 nm, preferably, 360 to 930 nm, and more preferably, 380 to 900 nm.

In addition, when measured with an X-ray photoelectron spectrometer, the surface of the porous PEEK implant, to which a collagen layer is not immobilized on the surface, may include nitrogen (N) in an amount of 4.00 to 6.00 atom%, preferably, 4.30 to 6.00 atom%, and more preferably, 4.50 to 5.50 atom%, based on 100 atom%of the total composition of carbon (C), oxygen (O), nitrogen (N) and sulfur (S) in the composition of the surface, and this N content is because nitro groups are generated on a portion of the surface of the implant during the etching process on the porous PEEK implant surface.

Next, the collagen layer will be described.

The collagen layer has excellent osseointegration ability, can accelerate bone formation, and performs a function of expressing an excellent effect of mineralized bone formation.

The collagen layer is immobilized on at least a portion of the PEEK implant surface, and preferably, the collagen layer is immobilized on all of the PEEK implant surface.

In addition, the collagen layer may be implemented through a fibrous stem formed by gathering a plurality of collagen molecules, and to this end, the collagen layer may be formed by using liquid collagen with increased solubility, and preferably, the collagen layer may be formed by using liquid collagen derived from pig skin.

In addition, the collagen layer may be formed of a porous collagen layer. If the reaction is performed by using solid collagen, for example, solid collagen derived from rat tail, the reactivity is lowered due to the low solubility of collagen such that it is difficult to form a collagen layer at a certain concentration.

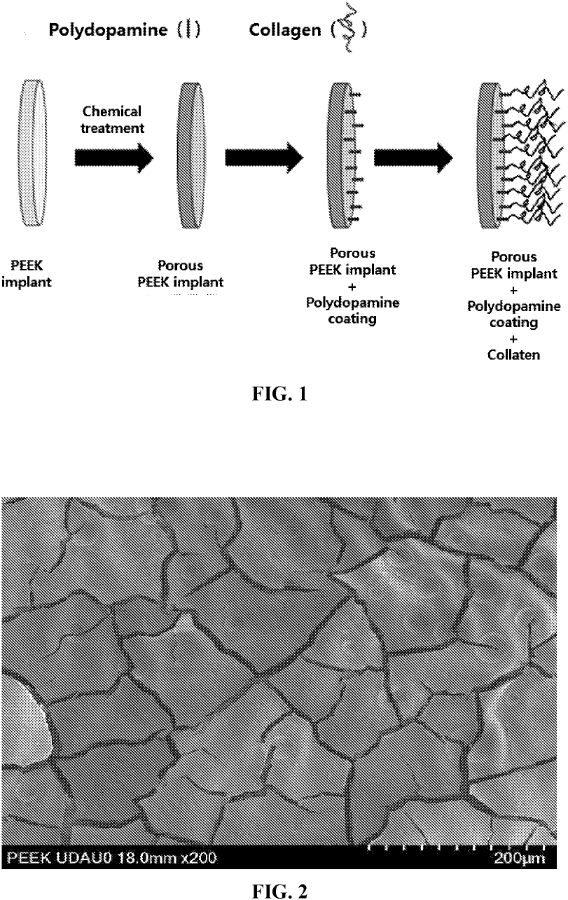

The collagen layer may be immobilized to the porous PEEK implant through polydopamine, and preferably, it may be immobilized by inducing mutual bonding with collagen through polydopamine coated on the porous PEEK implant surface, and more preferably, as illustrated in FIG. 1, the collagen layer may be immobilized to the PEEK implant by chemical bonding with the amine group of dopamine.

If a collagen layer is used by binding with an untreated porous PEEK implant, since there is no reactive group such as an amino group on the PEEK surface, the amount of the immobilized collagen layer is greatly limited, and thus, osseointegration is reduced, bone formation may not be accelerated, and mineralized bone formation may be reduced, and dissociation with the implant may occur. However, since the PEEK implant according to the present invention is capable of highly integrating the collagen layer to be described below by chemical bonding through dopamine, it is possible to simultaneously exhibit the effects of having excellent osseointegration, accelerating bone formation and having excellent mineralized bone formation, and dissociation with the implant does not occur.

When the surface of the porous biocompatible implant of the present invention formed with a collagen layer immobilized by polydopamine is measured with an X-ray photoelectron spectrometer, nitrogen (N) may be included in an amount of 15.50 atom% or more, preferably, 16.00 to 18.00 atom%, and more preferably, 16.30 to 17.40 atom%, based on 100 atom% of the total composition of carbon (C), oxygen (O), nitrogen (N) and sulfur (S).

The method for manufacturing the porous biocompatible implant of the present invention described above will be described below.

The porous biocompatible implant of the present invention may be manufactured by performing a process including step 1 of manufacturing a porous PEEK implant by forming a plurality of pores on the surface of a PEEK implant; step 2 of manufacturing a polydopamine-coated porous PEEK implant by coating and drying the porous PEEK implant with a dopamine coating solution; and step 3 of immobilizing a porous collagen layer on at least a portion of the surface of the polydopamine-coated porous PEEK implant.

The porous PEEK implant of step 1 is formed with pores on the surface by performing an etching process, and may be manufactured by two methods (method 1, method 2).

Method 1: It may be manufactured by performing step 1-1 of performing an immersion process by immersing the PEEK implant in a mixed acid solution; step 1-2 of performing stirring, after the PEEK implant subjected to the immersion process is placed into distilled water; step 1-3 of washing the PEEK implant for which step 1-2 has been performed with a high-pressure washer; and step 1-4 of drying the PEEK implant for which step 1-3 has been performed, after performing ultrasonic cleaning. As the mixed acid solution, an acid solution in which sulfuric acid having a concentration of 85.00 to 99.99% by volume and nitric acid having a concentration of 60 to 70% by volume are mixed at a weight ratio of 1 : 0.7 to 1.2 by weight, and preferably, an acid solution in which sulfuric acid having a concentration of 90.00 to 99.99% by volume and nitric acid having a concentration of 62.0 to 69.00 % by volume at a weight ratio of 1: 0.8 to 1.2 are mixed may be used, and more preferably, an acid solution in which sulfuric acid having a concentration of 95.00 to 99.99% by volume and nitric acid having a concentration of 64.0 to 68.00% by volume are mixed at a weight ratio of 1: 0.8 to 1.1 may be used. In this case, if the concentration of sulfuric acid and/or nitric acid is out of the above range, pores having a uniform size may not be formed on the surface of the PEEK implant. In addition, if the mixing ratio of nitric acid in the mixed acid solution is less than a weight ratio of 0.7, it is difficult for the pore layer to maintain a single layer, and pores may not be formed with a uniform size, and if it is more than a weight ratio of 1.2, the number of pores formed on the surface may be small.

In step 1-1, the immersion process may be performed by immersing the PEEK implant in the mixed acid solution for 1 to 5 minutes and preferably, for 1 to 3 minutes, and if the immersion time is out of 1 to 5 minutes, because the number of pores formed on the PEEK surface is too small or too large, the mechanical strength may be insufficient, and thus, it is preferable to perform the immersion process only during the above time.

In addition, step 1-2 of method 1 is a washing process for acid removal, and after immersing the PEEK implant subjected to the immersion process in distilled water, stirring may be performed at a stirring speed of 200 to 500 rpm, preferably, a stirring speed of 200 to 350 rpm for 3 minutes to 10 minutes.

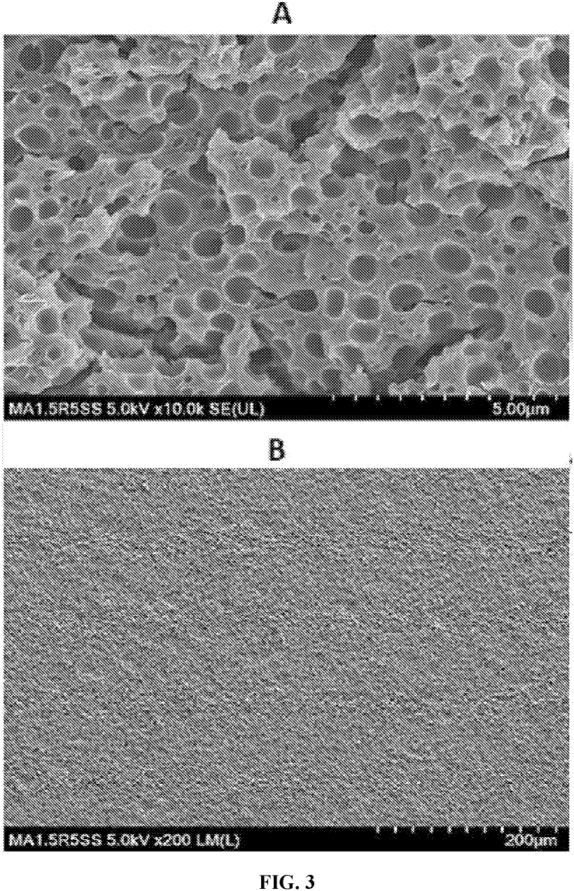

In addition, step 1-3 of method 1 is a process for removing cracked fragments on the surface of the PEEK implant as shown in FIG. 2 where steps 1-2 has been performed, and it is a process of peeling off cracked fragments from the surface of the implant by performing high-pressure cleaning by applying water at a water pressure of 120 to 170 bar for 0.5 minutes to 30 minutes and preferably, at a water pressure of 130 to 160 bar for 1 minute to 5 minutes by using a high-pressure washer (refer to A and B of FIG. 3). In this case, if the water pressure is less than 120 bar, the cracked fragments may not be removed well, and if it is more than 170 bar, the pores formed on the surface of the implant may be damaged, and thus, it is preferable to perform high-pressure washing at a water pressure within the above range.

In addition, step 1-4 of method 1 is an ultrasonic cleaning process for removing residual debris present on the surface of the PEEK implant and foreign substances blocking the pores, and after the PEEK implant for which the ultrasonic cleaning of method 1 and step 1-3 has been performed is placed in distilled water, ultrasonic cleaning may be performed for 5 to 120 minutes by applying an ultrasonic intensity of 15 to 80 KHz, preferably, an ultrasonic intensity of 15 to 50 KHz, and more preferably, an ultrasonic intensity of 15 to 30 KHz. In this case, if the ultrasonic intensity is less than 15 KHz, a large amount of occluded pores may remain on the surface of the implant, and if the ultrasonic intensity is more than 80 KHz, since the number of pores may be reduced and the pores may be formed non-uniformly on the surface, it is preferable to perform ultrasonic cleaning at an ultrasonic intensity within the above range (refer to A and B of FIG. 3).

In addition, the drying in steps 1-4 may be performed by a general drying method used in the art, and for example, natural drying or thermal drying at 50 to 70° C. may be performed.

Method 2: Method 2 of manufacturing a porous PEEK implant is a method omitting high-pressure washing, and it may be manufactured by performing step 1-1 of performing an immersion process under ultrasonication by immersing the PEEK implant in a mixed acid solution; step 1-2 of performing stirring, after the PEEK implant subjected to the immersion process is placed into distilled water; and step 1-3 of drying the PEEK implant for which step 1-2 has been performed, after performing ultrasonic cleaning.

In method 2, step 1-1 is different from step 1-1 of method 1 in that the mixed acid is treated under ultrasonication.

After the PEEK implant for which steps 1-2 has been performed is placed into distilled water, ultrasonic cleaning may be performed for 5 to 120 minutes at an ultrasound intensity of 15 to 80 KHz, preferably, an ultrasonic intensity of 40 to 80 KHz, and more preferably, 50 to 80 KHz. In this case, if the ultrasonic intensity is less than 30 KHz, the cracked fragments on the surface of the implant may be insufficiently removed, and a large amount of occluded pores may remain, and if the ultrasonic intensity is more than 80 KHz, rather, the periphery of the pores formed through the process of steps 1-1 to 1-2 is destroyed, the number of pores is reduced, and pores may be formed non-uniformly on the surface, and thus, it is preferable to perform ultrasonic cleaning at an ultrasonic intensity within the above range.

In addition, the drying in step 1-3 may be performed by a general drying method used in the art, and for a preferred example, natural drying at room temperature or thermal drying at 50 to 70° C. may be performed.

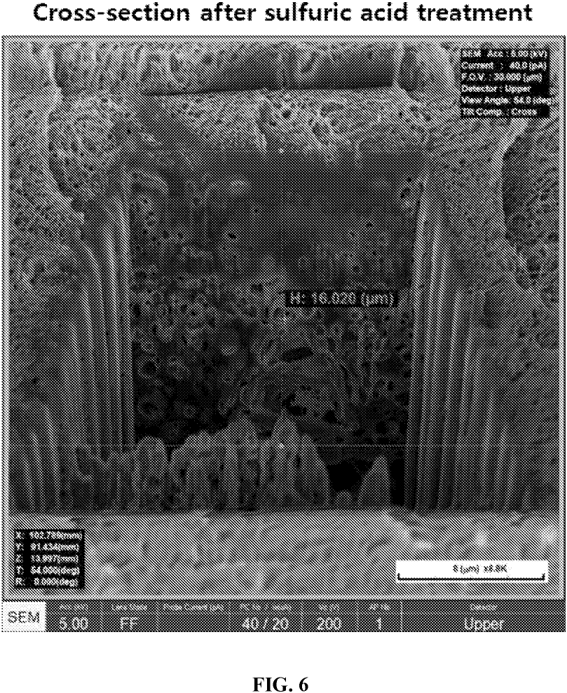

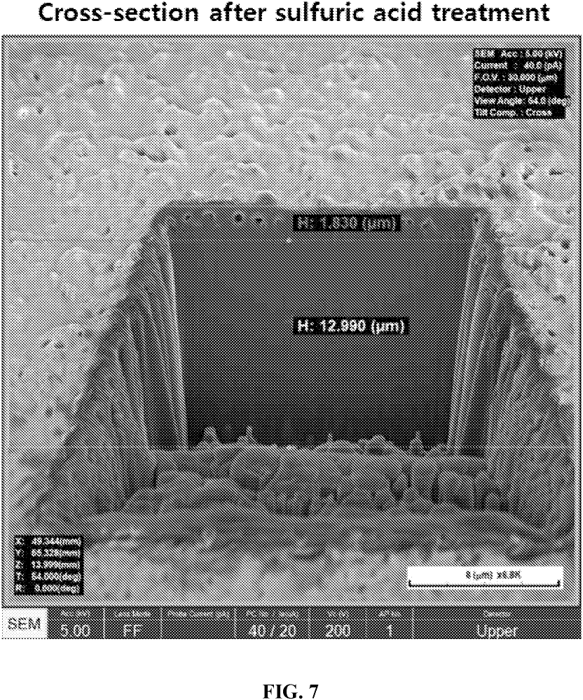

The porous PEEK implant in step 1 is subjected to an etching process by using a mixed acid (sulfuric acid/nitric acid) to form porosity on the surface. Meanwhile, it is also possible to form porosity on the surface by performing an etching process using a single acid (sulfuric acid). In the present invention, in order to compare the thicknesses of the pore layers of the single acid-treated and mixed acid-treated surfaces, the PEEK implant surface was etched in a V-shape using focused ion beams, and then observed with a scanning electron microscope, as shown in FIGS. 6 and 7.

FIG. 6 is a surface tomography image obtained by treatment with a single acid (sulfuric acid). In the case of a PEEK implant obtained by single acid treatment, as shown in D of FIG. 5, large and small pores are formed deep in multiple layers. Looking at the cross-section after etching in FIG. 6, it can be seen that a pore layer having a minimum depth of 16 µm or more was formed, and the etched surrounding tissues were collapsed. This is because sulfuric acid penetrated deep into the PEEK at a fast rate, and the pore density was too large to withstand the focused argon ion beams (FIB), and it collapsed.

FIG. 7 shows an image after etching of a PEEK implant treated with mixed acid using focused ion beams. It can be seen that the pore layer has a thickness of about 1.83 µm and forms a single layer. It can also be seen that the etched surrounding tissues are well maintained. From these results, it can be seen that by using a mixed acid of sulfuric acid/nitric acid for the PEEK implant, a porous PEEK implant with uniform pore size and pores formed in a single layer may be manufactured.

Next, step 2 of the method for manufacturing a porous biocompatible implant of the present invention performs a process of manufacturing a polydopamine-coated porous PEEK implant by coating the porous PEEK implant manufactured in step 1 by method 1 or method 2 described above with a dopamine coating solution and drying (refer to FIG. 1).

The coating method of step 2 is not particularly limited, such as spraying, immersing, rolling and the like, but for example, after immersing the porous PEEK implant in a polydopamine coating solution using an immersion method, coating may be performed by performing a roller mixer for about 10 to 30 hours, preferably, 20 to 28 hours.

In addition, the dopamine coating solution includes dopamine hydrochloride and a solvent, and the solvent may be used without limitation as long as it is a known solvent capable of dissolving dopamine, and it is preferable to use a Tris buffer solution having a pH of 8.0 to 9.0 (Tris(hydroxymethyl) aminomethane buffer solution).

In addition, the dopamine coating solution preferably includes the dopamine hydrochloride at a concentration of 0.05 to 0.40 mg/mL, and preferably, 0.25 to 0.40 mg/mL. In this case, if the concentration of dopamine hydrochloride is less than 0.05 mg/mL, the amount of polydopamine coated on the surface of the implant is small, and in the following process, the adhesion rate and immobilization rate of collagen are low, and thus, and problems such as poor osseointegration of porous bioimplants, decreased bone formation, dissociation with the implant may occur and the like. In addition, even if it is more than 0.40 mg/mL, there is no increase in the adhesion rate and immobilization rate of collagen anymore, and thus, it is preferable to include dopamine hydrochloride at a concentration within the above range.

In addition, the drying in step 2 may be performed by a general drying method used in the art, and for example, natural drying at room temperature or drying under a vacuum oven may be performed.

Next, step 3 of the method for manufacturing a porous biocompatible implant of the present invention is a process of immobilizing a porous collagen layer on at least a portion of the surface of the polydopamine-coated porous PEEK implant, and a collagen layer may be performed by coating, cleaning and drying the polydopamine-coated porous PEEK implant with a liquid collagen solution.

The coating method in step 3 is not particularly limited, such as spraying, dipping, rolling and the like, but for a preferred example, after immersing the polydopamine-coated porous PEEK implant in the liquid collagen coating solution using an immersion method, coating may be performed by performing a roller mixer for about 30 to 60 hours, preferably, 40 to 55 hours.

In addition, the liquid collagen solution may include collagen, 1-ethyl-3-(3-dimethyl aminopropyl)carbodiimide (EDC), N-hydroxysuccinimide (NHS) and water.

As the liquid collagen, commercially available liquid collagen may be used, and for a preferred example, salt-precipitated and compression-concentrated liquid collagen derived from pig skin may be used. In addition, the content of liquid collagen in the solution may include 0.0005 to 0.05 wt.%, preferably, 0.001 to 0.01 wt.%. In this case, if the content of liquid collagen in the total weight of the solution is less than 0.0005 wt.%, the desired level of effect may not be expressed because the amount of collagen to be immobilized is significantly low, and even if the content of liquid collagen is more than 0.05 wt.%, the amount of collagen introduced does not increase.

In addition, the EDC and NHS are to activate the carboxyl group of liquid collagen, and it is preferable to use each of EDC and NHS in an amount of 0.100 to 0.200 wt.% based on the total weight of the solution.

In step 3, the washing may be performed 1 to 3 times for the implant coated with liquid collagen with purified water or deionized water.

In addition, the drying in step 3 may be performed by a general method used in the art, and preferably, drying may be performed at room temperature under natural drying or a vacuum oven.

The porous biocompatible implant of the present invention described above may be used without limitation in the case of implants that are placed in vivo, such as for treatment and correction for fracture fixation, artificial spine, artificial prosthesis, orthodontic use and the like, and preferably, it may be used as an orthopedic implant.

The present invention will be described in more detail through the following examples, but the following examples are not intended to limit the scope of the present invention, and it should be construed to aid the understanding of the present invention.

### EXAMPLES

### Comparative Preparation Example 1: Manufacture of Surface-etched PEEK Implant

A spinal cage for intervertebral fusion prosthesis manufactured using a PEEK implant (VESTAKEEP^{®} i4 R, Manufacturer: Evonik Operations GmbH) was prepared.

Next, after immersing the PEEK implant in a mixed acid solution, stirring was performed for 30 minutes with a 250-rpm magnetic stirrer. In this case, the mixed acid solution was prepared by mixing 95% by volume of sulfuric acid and 65% by volume of nitric acid at a weight ratio of 1:1.

Next, after taking out the PEEK implant from the mixed acid, it was washed with distilled water for 1 minute and 30 seconds, and then immersed in 1,500 mL of distilled water, followed by stirring at a speed of 250 rpm for 5 minutes.

Next, after taking out the PEEK implant from distilled water, it was dried at about 25 to 26° C. for 24 hours to manufacture a surface-etched PEEK implant.

The SEM image of the manufactured PEEK implant is shown in FIG. 2.

Referring to FIG. 2, many cracks were formed on the surface of the PEEK implant, and it was confirmed that the cracked fragments were not peeled off.

### Preparation Example 1: Preparation 1 of Porous PEEK Implant

### Surface Etching Treatment Process

As a PEEK implant, a spinal cage for intervertebral fusion prosthesis identical to Comparative Preparation Example 1 was prepared.

Next, after immersing the PEEK implant in a mixed acid solution, stirring was performed for 30 minutes with a 250-rpm magnetic stirrer. In this case, the mixed acid solution was prepared by mixing 95% by volume of sulfuric acid and 65% by volume of nitric acid at a weight ratio of 1:1.

Next, after taking out the PEEK implant from the mixed acid, it was washed with distilled water for 1 minute and 30 seconds, and then immersed in 1,500 mL of distilled water, followed by stirring at a speed of 250 rpm for 5 minutes.

### High-Pressure Washing Process

Next, after taking out the PEEK implant from distilled water, about 150 bar of water pressure was applied to the surface of the PEEK implant using a high-pressure washer, and high-pressure washing was performed for 1 to 5 minutes.

### Ultrasonic Cleaning and Drying Process

Next, after the PEEK implant that had been subjected to the high-pressure washing process was placed into distilled water, ultrasonic cleaning was performed for 60 minutes by applying an ultrasonic intensity of 30 KHz.

Next, the ultrasonically cleaned PEEK implant was placed into a vacuum dryer and then dried at 60° C. for 24 hours to manufacture a porous PEEK implant.

The SEM images of the manufactured porous PEEK implant are shown in A and B of FIG. 3.

Referring to FIG. 3, it was confirmed that, unlike Comparative Preparation Example 1, there was no crack fragment on the surface, and a two-dimensional pore structure was uniformly formed on the surface of the implant.

### Preparation Example 2: Preparation 2 of Porous PEEK Implant

A PEEK implant that had been subjected to a surface etching process in the same manner as in Preparation Example 1 was manufactured.

Next, after placing the PEEK implant in distilled water, ultrasonic cleaning was performed for 1 to 5 minutes by applying an ultrasonic intensity of 60 KHz.

Next, the ultrasonically cleaned PEEK implant was placed into a vacuum dryer and then dried at 60° C. for 24 hours to manufacture a porous PEEK implant.

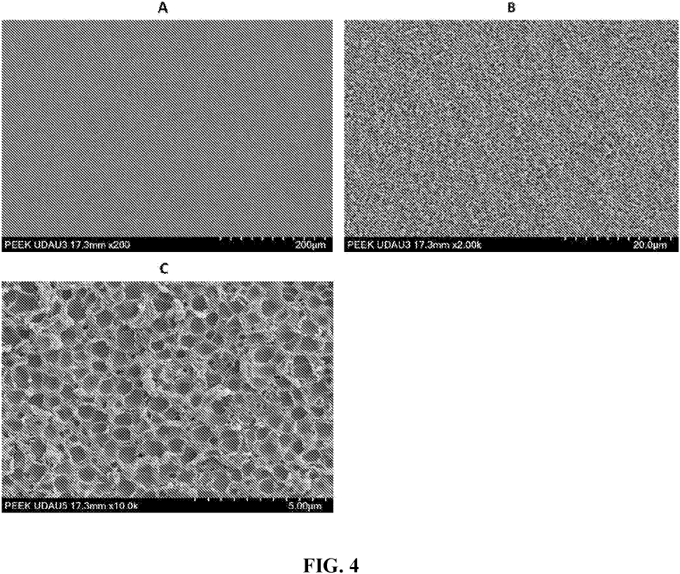

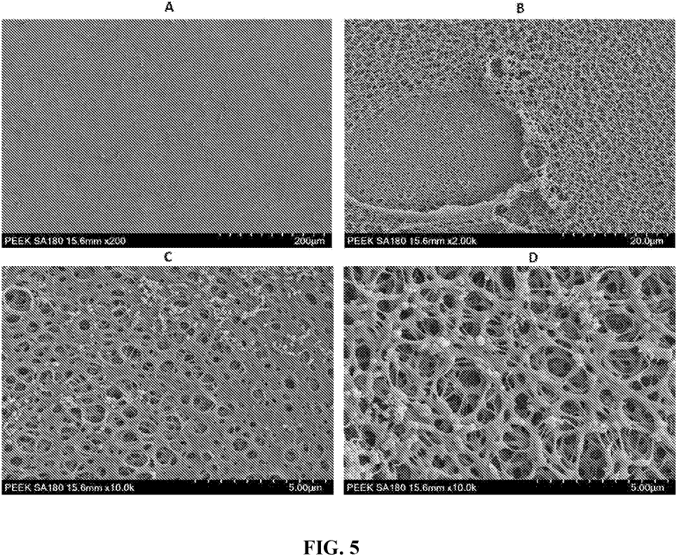

The SEM images of the manufactured porous PEEK implant are shown in A and C of FIG. 4.

Referring to FIG. 4, it was confirmed that, unlike Comparative Preparation Example 1, there was no crack fragment on the surface, and a two-dimensional pore structure was uniformly formed on the surface of the implant.

### Comparative Preparation Example 2: Preparation of Porous PEEK Implant

As a PEEK implant, a spinal cage for intervertebral fusion prosthesis identical to Comparative Preparation Example 1 was prepared.

Next, after the PEEK implant was immersed in a sulfuric acid solution having a concentration of 99.5% by volume, it was immersed for 30 minutes, followed by ultrasonic washing for 15 minutes each with acetone, ethanol and distilled water.

Afterwards, it was dried at 120° C. for 4 hours to manufacture an etched PEEK implant having a three-dimensional structure.

The SEM images of the manufacture porous PEEK implant are shown in A to C of FIG. 5. Looking at the low magnification SEM image of A of FIG. 5 and the medium magnification SEM image of B in FIG. 5, it was confirmed that pores were formed on a nonuniform surface, and several hollow lungs were formed in the shape of a well. In addition, C is an SEM image of a part (red square) of the left hollow lung of B, and D is an SEM image of a part (white square) of the right part measured at high magnification, and it was confirmed that the pore layer was formed in a multi-layered structure deep from the surface of the PEEK implant.

### Experimental Example 1: Measurement of Surface Pore Size and Pore Layer Thickness

For the porous PEEK implants manufactured in Preparation Examples 1 and 2 and Comparative Preparation Example 2, the surface was etched in a V shape with focused ion beams (FIB), and the average size and pore layer thickness of the pores formed on the surface were measured by SEM, and the results are shown in Table 1 below.

In addition, after etching Comparative Preparation Example 2 in a V shape, an image observed with a scanning electron microscope is shown in FIG. 6, and after etching Preparation Example 2 into a V shape, an image observed with a scanning electron microscope is shown in FIG. 7.

Table 1

| Classification | Pore size (nm) | Average thickness of pore layer (µm) |
|---|---|---|
| Preparation Example 1 | 380 ~ 900 | About 1.90 |
| Preparation Example 2 | 390 ~ 900 | About 1.75 |
| Comparative Preparation Example 2 | 180 ∼ 2,300 | About 22.51 |

As can be confirmed in FIGS. 3 and 4, in the porous PEEK implants manufactured in Preparation Examples 1 and 2, pores were uniformly and evenly formed on the surface of the PEEK implant, and it could be confirmed that as shown in FIG. 7 by these characteristics, the thickness of the surface pore layer ranged from 0.5 to 3 µm, indicating a single layer, and the pore size was less than 1000 nm, preferably, about 360 to 930 nm. In particular, in Preparation Examples 1 and 2, a pore layer having a single-layer structure was formed with a very low thickness from the surface, and pores having a two-dimensional structure were formed.

In contrast, as can be confirmed in FIGS. 5 and 6, in Comparative Preparation Example 2 treated with sulfuric acid, a large amount of non-uniformly sized pores were formed, and the pores were formed from the surface to the deep portion in a three-dimensional structure, and the pore layer thickness showed a very large average thickness of about 22.5 µm, compared to Preparation Examples 1 and 2. In addition, the porous PEEK implant of Comparative Preparation Example 2 exhibited a relatively large pore size of 180 to 2,300 nm, compared to Preparation Examples 1 and 2.

### Experimental Example 2: Measurements of Tensile Strength and Compressive Strength

The tensile strength and compressive strength of each of the porous PEEK implants of Preparation Examples 1 to 2 and Comparative Preparation Example 2 were measured, and the results are shown in Tables 2 to 3, respectively.

Tensile strength measurement was performed by manufacturing standard dumbbell-type specimens according to Section E 5 by the tensile strength measurement method (ASTM D638) of PEEK used for orthopedic implants presented by the Food and Drug Safety Evaluation Institute of the Ministry of Food and Drug Safety. The tensile strength tester was performed at a temperature of 24° C. and a humidity of about 35% by using model ST-1002 (Manufacturer: SALT Co., Ltd.). The control group in Table 1 below is a result of tensile strength measurement for the PEEK implant used in Preparation Example 1 in which the surface has not been not etched with acid, and the average values of the measurement after measuring 12 samples are shown in Table 2 below.

Table 2

| Classification | Tensile strength (kgf/mm²) | Loss rate of tensile strength compared to control group (%) |
|---|---|---|
| Control group | 15.078 | 0 |
| Preparation Example 1 | 14.913 | 1.1 |
| Preparation Example 2 | 14.976 | 0.7 |
| Comparative Preparation Example 2 | 14.897 | 1.2 |

Looking at the tensile strength measurement results in Table 2, it was confirmed that Preparation Examples 1 and 2 prepared by etching the surface with a mixed acid, and Comparative Preparation Example 2 prepared by treatment with a single acid (sulfuric acid) had almost similar tensile strengths compared to the control group without acid treatment.

Table 3

| Classification | Compressive strength (N) | Loss rate of compressive strength compared to control group (%) |
|---|---|---|
| Control group | 15,168 | 0 |
| Preparation Example 2 (mixed acid) | 15,130 | 0.25 |
| Comparative Preparation Example 2 (single acid) | 15,123 | 0.29 |

Table 3 shows the compressive strengths of the porous PEEK implants obtained by treatment with a mixed acid and a single acid. It was confirmed that both of Preparation Example 2 prepared by etching the surface with a mixed acid and Comparative Preparation Example 2 prepared by treatment with a single acid (sulfuric acid) had almost similar compressive strengths, compared to the control group without acid treatment. It was found that even when the acid was treated to form pores on the surface of the PEEK implant, the tensile strength and compressive strength of the sample were not significantly affected.

### Example 1: Preparation of Porous Biocompatible Implant

The porous PEEK implant manufactured in Preparation Example 2 above was prepared.

Separately, 1.214 g of Trizma hydrochloride was dissolved in 100 mL of distilled water to prepare 10 mmol of a Tris buffer solution (Tris (hydroxymethyl) aminomethane buffer solution). In addition, after adjusting the Tris buffer solution to pH 8.5, 30 mg of dopamine hydrochloride was added thereto, stirred and dissolved to prepare a dopamine coating solution containing 0.30 mg/mL of dopamine hydrochloride.

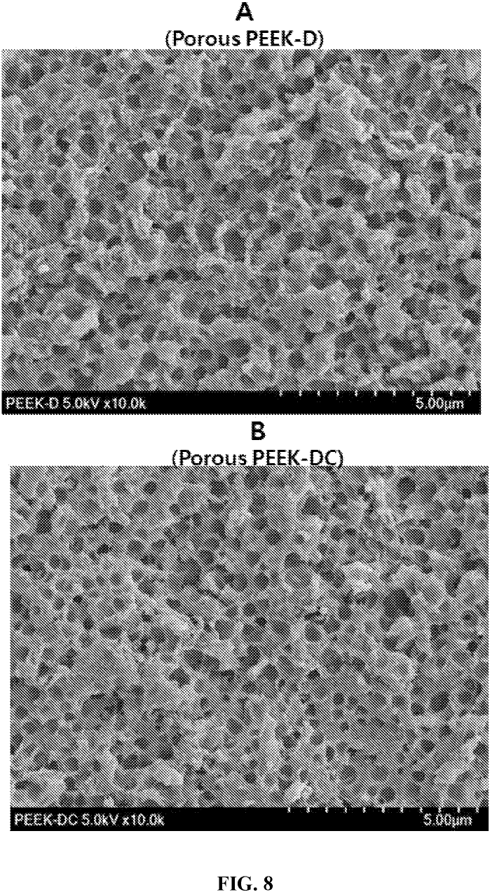

After loading the porous PEEK implant of Preparation Example 2 in the dopamine coating solution, a roller mix was performed for 24 hours to coat the surface of the implant with polydopamine. Afterwards, after washing with water, it was placed into a vacuum dryer and dried at about 25 to 26° C. for 24 hours to manufacture a polydopamine-coated porous PEEK implant (porous PEEK-D). The SEM measurement image of the manufactured porous PEEK-D is shown in A of FIG. 8.

Next, the porous PEEK-D was immersed in the collagen solution, and then a roller mixer was performed for 48 hours. In this case, for the collagen solution, after dissolving 0.125 g of EDC (1-ethyl-3-(3-dimethyl aminopropyl) carbodiimide) and 0.125 g of NHS (N-hydroxysuccinimide) in distilled water per 100 g (or mL) of distilled water, a solution containing 0.1 wt.% of salt-precipitated compression-concentrated liquid collagen derived from pig skin dissolved in distilled water was used.

Next, the porous PEEK-D was taken out from the collagen solution, washed with distilled water 3 times, and then placed into a vacuum dryer and dried at about 25 to 26° C. for 24 hours to manufacture a porous biocompatible implant (porous PEEK-DC) in which a collagen layer was immobilized.

In addition, the SEM measurement image of the manufactured porous PEEK-DC is shown in B of FIG. 8.

Referring to A of FIG. 8, it was confirmed that the porous PEEK-D obtained by polymerizing coating polydopamine on the surface of the porous PEEK implant manufactured by surface etching with a mixed acid maintained the pore structure well. In addition, referring to B of FIG. 8, it was confirmed that the porous PEEK-DC to which collagen was bound through polydopamine also maintained pores well.

As described above, the porous biocompatible implant of the present invention having two-dimensional pores on the surface not only increases the collagen introduction rate by significantly increasing the specific surface area, but also the PEEK having a pore structure with a large specific surface area increases the contact area with the bone tissue, and thus, it is possible to increase the binding strength and biocompatibility by collagen.

### Comparative Example 1

A biocompatible implant was manufactured in the same manner as in Example 1, except that instead of the porous PEEK implant of Preparation Example 2, a pore-free PEEK implant whose surface was not acid-treated (etched) (VESTAKEEP^{®} i4 R, Manufacturer: Evonik Operations GmbH) was used to manufacture a biocompatible implant by coating polydopamine and collagen on the surface of the PEEK implant by using the same method and conditions.

### Examples 2 to 3 and Comparative Examples 2 to 3

After manufacturing porous implants in the same manner as in Preparation Example 2, porous biocompatible implants were manufactured in the same manner as in Example 1 by using the same. However, when PEEK-D was manufactured, solutions in which the concentrations of dopamine hydrochloride in the dopamine coating solution were prepared to be 0.20 mg/mL (Example 2) and 0.40 mg/mL (Example 3), respectively, were used to respectively manufacture porous biocompatible implants, and Example 2 and Example 3 were carried out.

In addition, after manufacturing porous implants in the same manner as in Preparation Example 2, porous biocompatible implants were manufactured in the same manner as in Example 1 by using the same. However, when PEEK-D was manufactured, solutions in which the concentrations of dopamine hydrochloride in the dopamine coating solution were 0.10 mg/mL (Comparative Example 2) and 0.50 mg/m: (Comparative Example 3), respectively, were used to respectively manufacture porous biocompatible implants, and Comparative Example 2 and Comparative Example 3 were carried out, respectively.

### Examples 4 to 6 and Comparative Example 5

After manufacturing porous implants in the same manner as in Preparation Example 2, porous biocompatible implants were manufactured in the same manner as in Example 1 by using the same.

However, when PEEK-D was manufactured, a collagen solution in which the concentration of dopamine in the dopamine coating solution was fixed to 0.3 mg/mL was used, and when PEEK-DC was manufactured, collagen solutions in which the concentrations of collagen were 0.01 wt.% (Example 4), 0.05 wt.% (Example 5) and 0.5 wt.% (Comparative Example 4) were used to respectively manufacture porous biocompatible implants, and Examples 4 to 5 and Comparative Example 4 were carried out, respectively.

### Experimental Example 3: XPS Measurement of Implant Surface

When the porous biocompatible implant of Example 1 and the biocompatible implant of Comparative Example 1 were manufactured, the surface of the implant for each implant in each process was analyzed with X-ray Photoelectron Spectroscopy (XPS), and the results are shown in Table 4 below.

Table 4

| Classification | | Atomic composition (atom%) | | | |
|---|---|---|---|---|---|
| | | C | O | N | S |
| Comparative Example 1 | PEEK | 85.89 | 13.04 | 0.59 | 0.48 |
| | PEEK-D | 74.71 | 18.46 | 6.71 | 0.12 |
| | PEEK-DC | 70.12 | 19.61 | 10.71 | 0.14 |
| Example 1 | Porous PEEK | 74.65 | 20.31 | 4.91 | 0.13 |
| | Porous PEEK-D | 71.10 | 20.22 | 8.57 | 0.11 |
| | Porous PEEK-DC | 63.34 | 19.83 | 16.69 | 0.14 |
| Example 2 | Porous PEEK-DC | 71.31 | 19.21 | 9.41 | 0.07 |
| Example 3 | Porous PEEK-DC | 70.42 | 17.05 | 12.32 | 0.21 |
| Comparative Example 2 | Porous PEEK-DC | 67.12 | 18.50 | 14.23 | 0.15 |
| Comparative Example 3 | Porous PEEK-DC | 66.25 | 19.34 | 14.28 | 0.13 |
| Example 4 | Porous PEEK-DC | 66.77 | 18.06 | 15.03 | 0.14 |
| Example 5 | Porous PEEK-DC | 66.76 | 19.12 | 13.97 | 0.15 |
| Comparative Example 4 | Porous PEEK-DC | 67.22 | 19.68 | 12.97 | 0.13 |

PEEK-D means that the surface of the PEEK implant is coated with polydopamine, and PEEK-DC means that the PEEK implant is coated with polydopamine and collagen.

In Table 4, when compared with the PEEK of Comparative Example 1 and the porous PEEK of Example 1, it can be confirmed that N appears newly on the surface of the PEEK implant. This is because nitration occurred at the benzyl group of PEEK during the treatment of a mixed acid of sulfuric acid/nitric acid to form surface pores.

In addition, when the N contents of PEEK-D and PEEK-DC of Comparative Example 1 and the porous PEEK-D and PEEK-DC of Example 1 are compared, it can be confirmed that the N content of Example 1 was relatively significantly increased compared to that of Comparative Example 1, which was because the polydopamine coating amount increased by pore formation and the collagen coating increased thereby. Meanwhile, when the collagen concentration was increased (Comparative Examples 3 and 4) or decreased (Comparative Example 2), a large change in the surface N content was not observed. This can be said to be unaffected by the concentration of collagen because the reaction sites where collagen could bind on the surface were limited.

### Experimental Example 4: Evaluation of Cell Compatibility of Implants (in Vitro)

In order to evaluate the cellular compatibility of the PEEK implant, PEEK (non-porous PEEK) of Comparative Example 1 without formation of pores, porous PEEK manufactured in Preparation Example 1 with pores formed, and the collagen-immobilized PEEK implant of Example 1 (porous PEEK-DC) were manufactured.

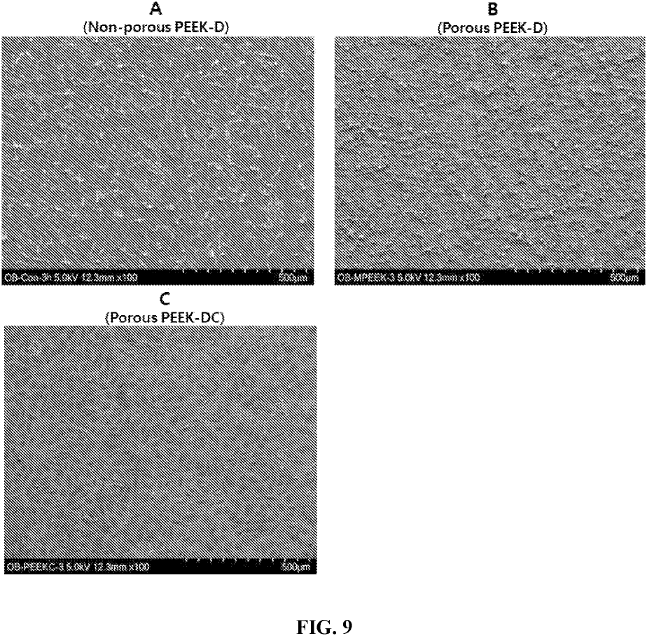

The osteoblasts were adjusted to 5 × 10³ /mL, and the adherent cells after 3 hours of culture were formalin-fixed and the images were observed by SEM, and the results are shown in A to C of FIG. 9. As a result, it can be seen that more cells were attached to the porous PEEK implant (B) than to the non-porous PEEK implant (A). From this, it can be seen that the nanopores on the surface promote the cell adhesion rate. Meanwhile, it was found that the most cells were attached to the porous PEEK-DC with collagen immobilized on the porous PEEK implant, and most of the cells performed well in spreading despite the short incubation time of 3 hours. This is because there is an Arg-Gly-Asp (RGD) sequence in the collagen molecule immobilized on the surface, and it is a result of the specific ligand/receptor interaction that is performed by integrin present in the osteoblast membrane recognizing the RGD sequence in collagen.

### Experimental Example 5: Calcium Production Promoting Effect of Implants (in Vitro)

In order to evaluate the cellular compatibility of the PEEK implant, PEEK (non-porous PEEK) of Comparative Example 1 without formation of pores, porous PEEK manufactured in Preparation Example 1 with pores formed, and the collagen-immobilized PEEK implant of Example 1 (porous PEEK-DC) were manufactured.

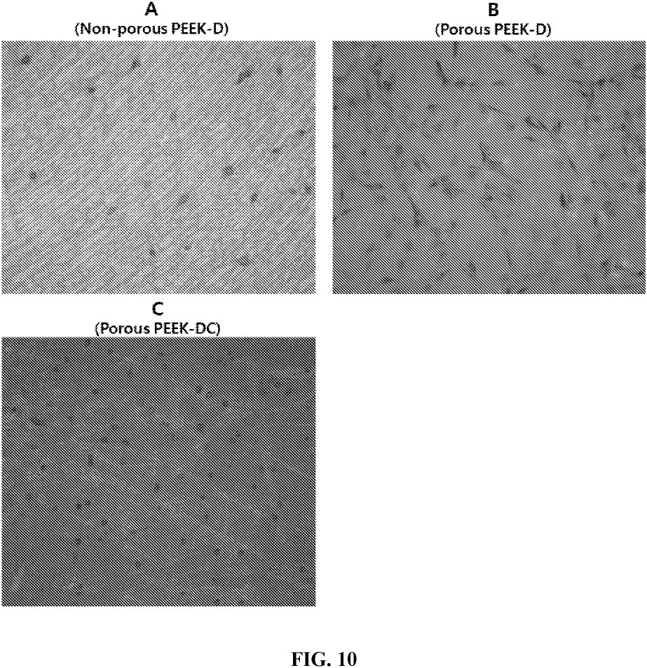

The osteoblasts were adjusted to 5 × 10³ /mL and cultured for 7 days, the medium was replaced every 3 days, and after the cells cultured for 7 days were formalin-fixed, alizarin Red S solution was added thereto, and it was left at room temperature for 1 hour. The fluorescence image was observed by SEM, and the results are shown in A to C of FIG. 10. As a result, more yellowish red color was developed in the porous PEEK implant (B) than in the non-porous PEEK implant (A), which means that calcium production was relatively promoted more in the porous PEEK than in the non-porous PEEK. In addition, it can be confirmed that this trend was more pronounced in porous PEEK-DC, and from this, it can be confirmed that nanopores and collagen immobilization on the PEEK surface have a very positive effect on the differentiation ability of cells for bone generation.

Although an exemplary embodiment of the present invention has been described above, the spirit of the present invention is not limited to the exemplary embodiment presented in the present specification, and those skilled in the art who understand the spirit of the present invention will be able to easily suggest other exemplary embodiments by modifying, changing, deleting or adding components within the scope of the same spirit, but this is also said to be within the scope of the present invention.

1. A porous biocompatible implant with excellent osseointegration, comprising:
a collagen layer which is immobilized on at least a portion of the surface of a porous polyether ether ketone (PEEK) implant,
wherein the porous PEEK implant is formed with a plurality of pores in a single layer on the surface.

2. The porous biocompatible implant of claim 1, wherein the porous PEEK implant is formed with a single pore layer having a thickness of 0.5 µm to 3 µm from the surface.

3. The porous biocompatible implant of claim 2, wherein after etching the surface of the implant in a V-shape with focused ion beams (FIB), the porous PEEK implant has a diameter of pores formed on the surface of less than 1,000 nm, when the cross-section is observed with a scanning electron microscope (SEM).

4. The porous biocompatible implant of claim 1, wherein the collagen layer is implemented through a stem on fibers formed by gathering a plurality of collagen molecules.

5. The porous biocompatible implant of claim 1, wherein the collagen layer is immobilized to the PEEK implant through polydopamine.

6. The porous biocompatible implant of claim 1, wherein the collagen layer includes those formed by using salt-precipitated compression-concentrated liquid collagen derived from pig skin.

7. The porous biocompatible implant of claim 1, wherein when the surface of the porous PEEK implant is measured with an X-ray photoelectron spectrometer, nitrogen (N) is included at 4.00 to 6.00 atom%based on 100% of the total composition of carbon (C), oxygen (O), nitrogen (N) and sulfur (S) in the composition of the surface.

8. The porous biocompatible implant of claim 1, wherein when the surface of the porous biocompatible implant is measured with an X-ray photoelectron spectrometer, nitrogen (N) is included at 15.50 atom%or more based on 100 atom% of the total composition of carbon (C), oxygen (O), nitrogen (N) and sulfur (S) in the composition of the surface.

9. A method for manufacturing a porous biocompatible implant with excellent osseointegration, comprising:
step 1 of manufacturing a porous PEEK implant by forming a plurality of pores on the surface of a PEEK implant;
step 2 of manufacturing a polydopamine-coated porous PEEK implant by coating and drying the porous PEEK implant with a dopamine coating solution; and
step 3 of immobilizing a porous collagen layer on at least a portion of the surface of the polydopamine-coated porous PEEK implant.

10. The method of claim 9, wherein the porous PEEK implant of step 1 is manufactured by performing:
step 1-1 of performing an immersion process by immersing the PEEK implant in a mixed acid solution for 1 minute to 5 minutes;
step 1-2 of performing stirring at a speed of 200 to 500 rpm for 3 minutes to 10 minutes, after the PEEK implant subjected to the immersion process is placed into distilled water;
step 1-3 of washing the PEEK implant for which step 1-2 has been performed with a high-pressure washer by applying water at a water pressure of 120 to 170 bar for 1 minute to 30 minutes; and
step 1-4 of drying the PEEK implant for which step 1-3 has been performed, after performing ultrasonic cleaning under an ultrasonic intensity of 15 to 50 KHz.

11. The method of claim 9, wherein the porous PEEK implant of step 1 is manufactured by performing:
step 1-1 of performing an immersion process under ultrasonication by immersing the PEEK implant in a mixed acid solution for 1 minute to 5 minutes;
step 1-2 of performing stirring at a speed of 200 to 500 rpm for 3 minutes to 10 minutes, after the PEEK implant subjected to the immersion process is placed into distilled water; and
step 1-3 of drying the PEEK implant for which step 1-2 has been performed, after performing washing under an ultrasonic intensity of 50 to 80 KHz.

12. The method of claim 9, wherein the dopamine coating solution includes dopamine hydrochloride and a Tris buffer aqueous solution having a pH of 8.0 to 9.0, and
wherein the dopamine coating solution includes dopamine hydrochloride at a concentration of 0.20 to 0.40 mg/mL.

13. The method of claim 9, wherein in step 3, the polydopamine-coated porous PEEK implant is coated with a liquid collagen solution, washed and dried to form a collagen layer, and
wherein the liquid collagen solution includes 0.0005 to 0.05 wt.% of collagen, 0.100 to 0.300 wt.% of EDC ((1-ethyl-3-(3-dimethyl aminopropyl)carbodiimide), 0.100 to 0.300 wt.% of NHS (N-hydroxy succinimide) and the remaining amount of water.
